(19) United States
(12) Patent Application Publication
(10) Pub. No.: US 2025/0308703 A1
(43) Pub. Date: Oct. 2, 2025

(54) METHODS AND SYSTEMS FOR METABOLIC TESTING

(71) Applicant: Sequelae, Inc., Minneapolis, MN (US)

(72) Inventors: Douglas A. Granger, Coto de caza, CA (US); Steve Granger, Encinitas, CA (US); Kiersten Clements, Murray, UT (US); James LaBelle, Encinitas, CA (US); Benjamin C. Wiegand, Yardley, PA (US); Steven J. Catani, Athens, GA (US)

(73) Assignee: Sequelae, Inc., Minneapolis, MN (US)

(21) Appl. No.: 19/091,565

(22) Filed: Mar. 26, 2025

Related U.S. Application Data
(60) Provisional application No. 63/569,984, filed on Mar. 26, 2024.

(51) Int. Cl. G16H 50/30; G16H 10/20

(52) U.S. Cl. CPC G16H 50/30; G16H 10/20

(57) ABSTRACT

To measure a member response in an individual member, a first resulting test point is measured before an activity. Then, at least a second resulting test point is measured at or after the activity. The member response can then be calculated as a function of the first resulting test point and the second resulting test point at the different times.

### CROSS-REFERENCE TO RELATED APPLICATIONS

This application claims the benefit of U.S. application Ser. No. 63/569,984, filed on Mar. 26, 2024, which is incorporated herein by reference in its entirety.

### BACKGROUND

Various embodiments relate generally to health care systems, methods, devices, computer programs, and more specifically, relate to biosample based testing such as metabolic testing.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Biosample based testing such as metabolic testing can be used to determine how an individual member's body works by measuring the ability of the individual member's body to use oxygen to produce energy. Such metabolic testing often relies on a single test point resulting from the test. However, the resulting test point will vary with the individual member's circadian rhythms, environment, food intake, exercise, level of stress, anxiety, happiness, and social context. Due to these factors, testing is typically taken at a controlled time and setting (e.g., in a fasting state in the morning).

Additionally, computers have changed the way people interact. Digital networks, which may include the use of social media, allow individuals to interact with others online and connect many people with a community. The online community can be utilized to help build positive behaviors and encourage people to make improvements in their lives.

What is needed is a way to build upon the social media and the access computers have in order to build healthy lifestyles, and develop healthy behaviors, as well as improving techniques to conduct metabolic testing.

### SUMMARY

Example aspects of the present disclosure include:

A method to determine a member response in an individual member according to at least one embodiment of the present disclosure comprises determining a member response by: receiving a first series of resulting test points including a first resulting test point measured prior to an activity and a second resulting test point measured after the activity; receiving a second series of resulting test points including a first resulting test point measured prior to an activity and a second resulting test point measured after the activity; and determining the member response as a function of the first series of resulting test points and the second series of resulting test points; determining an intervention based on the member response; providing the intervention to the individual member; determining a change in the individual member over a plurality of iterations of determining the member response; and determining an efficacy of the intervention based on the change in the member response over the plurality of iterations.

Any of the aspects herein, wherein the first resulting test point and the second resulting test point comprise a first insulin level and a second insulin level, respectively.

Any of the aspects herein, wherein the first series of resulting test points and the second series of resulting test points are measured using a continuous glucose monitor.

Any of the aspects herein, wherein the activity comprises instructing the individual member to ingest at least one of food or a fluid.

Any of the aspects herein, wherein the food or the fluid comprises a mixture of a glucose moiety.

Any of the aspects herein, wherein the member response is calculated as a function of the difference between the first resulting test point and the second resulting test point.

Any of the aspects herein, further comprising receiving a third resulting test point, and wherein the member response is determined as a function of the time taken for the third stress resulting test point to return to the range of the first resulting test point.

Any of the aspects herein, wherein the member response is determined as a function of a slope of the second resulting test point and the third stress indicator level.

Any of the aspects herein, wherein the member response is calculated as an index of a function of the difference between the first resulting test point and both the second resulting test point and the third resulting test point, the time it takes for a fourth resulting test point taken after the third resulting test point to return to the range of the first resulting test point, and a function of a slope of both the second resulting test point and the third resulting test point.

Any of the aspects herein, wherein measuring the first resulting test point and the second resulting test point comprises taking a reading using at least one of: a psychometric instrument, a heart rate monitor, and a pulse oximeter.

Any of the aspects herein, wherein measuring the first resulting test point and the second resulting test point comprises sharing the first resulting test point and the second resulting test point using the DCN.

A method to determine member response in an individual member according to at least one embodiment of the present disclosure comprises determining the member response by: receiving a first resulting test point measured prior to an activity; receiving at least a second resulting test point and a third stress indicator level measured at two or more time points after the activity, wherein the activity is operable to induce a stress response; and determining the member response as a function of the first measured stress indicator level, the second measured stress indicator level, and the third measured stress indicator level, wherein the first resulting test point, the second resulting test point, and the third stress indicator level form a plurality of stress indicator levels; determining an intervention for an intervention based on the member response; and providing the intervention to a user.

Any of the aspects herein, wherein the method is performed at multiple instances, and wherein the method further comprising determining a change in the member response over multiple iterations of the method.

Any of the aspects herein, further comprising determining an efficacy of an intervention based on the change in the member response over the multiple instances.

Any of the aspects herein, wherein the member response is calculated as a function of the difference between the first resulting test point and both the second resulting test point and the third resulting test point.

Any of the aspects herein, wherein the member response is determined as a function of the time taken for a fourth resulting test point taken after the third resulting test point to return to the range of the first resulting test point.

Any of the aspects herein, wherein the member response is determined as a function of a slope of the second resulting test point and the third resulting test point.

A system according to at least one embodiment of the present disclosure comprises a computer processor; a data repository in communication with the computer processor and storing: individual statistics having a member response and historic readings, resulting test point data having at least a first resulting test point and a second resulting test point, an activity, and an intervention; an activity controller which, when executed by the computer processor, administers the activity; a member response generator which, when executed by the computer processor, determines the member response; a digital communications network which, when executed by the computer processor, provides a network for members of a population to interact with each other and for an individual member to access a metabolic test for determining the individual member's member response; a server controller which, when executed by the computer processor: determines the member response by: receiving a first series of resulting test points including a first resulting test point obtained prior to the activity and a second resulting test point after the activity, receiving a second series of resulting test points including a first resulting test point obtained prior to the activity and a second resulting test point after the activity, determining a member response as a function of the first series of resulting test points and the second series of resulting test points, determines an intervention for an individual based on the member response; provides the intervention to the user; determines a change in the member response over a plurality of iterations of determining the member response; and determines an efficacy of the intervention based on the change in the member response over the plurality of iterations.

Any of the aspects herein, wherein the first resulting test point and the second resulting test point are a first insulin level and a second insulin level.

Any of the aspects herein, wherein the activity comprises instructing the individual member to ingest at least one of food or a fluid.

### BRIEF DESCRIPTION OF THE DRAWINGS

Aspects of the described embodiments are more evident in the following description, when read in conjunction with the attached Figures.

FIG. 1 shows a simplified block diagram of devices in accordance with various embodiments.

FIG. 2 is a logic flow diagram that illustrates a method in accordance with various embodiments.

FIG. 3A shows an example of a computing system, in accordance with one or more embodiments.

FIG. 3B shows an example of a network, in accordance with one or more embodiments.

### DETAILED DESCRIPTION

Metabolic testing can be enhanced by evaluating a resulting test point before and after a known activity that influences the resulting test point. This improves the understanding of both the baseline and dynamic aspects of an individual member's resulting test point and can improve the signal-to-noise of the analysis. Further standardizing aspects of the metabolic testing (such as time) also enables test results to be compared between individuals. Rather than using the metabolic test to compare different people to each other, metabolic testing can be used to determine if an intervention worked for an individual member. Accordingly, various embodiments provide a method that allows the individual member to select an activity that is easy for them to do and is optimized for self-improvement instead of medical decision making.

The subjective experience of an individual member's resulting test point as determined from metabolic testing, rather than the objective features of the activity(s) themselves, can assist in understanding an individual member's differences between the resulting test point before, during, and/or after the activity. The association between a behavior and biology is also dependent on the context. For example, measurements taken in the context of standardized conditions are designed to represent key features that are deemed to have a high value (e.g., “labs” obtained using single measures collected during clinic visits). However, such standardized conditions can remove the potential influence of other factors that are seen to have a lower value. Here, “high value” refers to the idea that a variation in these parameters is deemed more likely to predict individual differences in the member's health and are of primary interest.

In at least one embodiment, metabolic testing can be taken relative to an activity, such as, playing a game, walking a mile, ingesting an ingestible product (e.g., food or fluids), or experiencing some sort of stimulus. The testing can be done before, during, and after the activity (as well as multiple times after the activity, such as, immediately after and 1 hour after the activity). The data is recorded, stored, and analyzed to gauge the effects of stimuli, or other factors (time of day, food/no food, etc.), on medical health, treatment, etc.

In at least one example of the above metabolic testing, the individual may imbibe an ingestible composition (e.g., a beverage comprising a glucose moiety). In another example, an individual performs an exercise activity (e.g., jogging on a treadmill, rowing, biking, swimming, etc.). During the exercise activity, the individual wears a mask which captures the air breathed in and out as the patient moves through various intensities of exercise, gradually increasing in difficulty. In some embodiments, the individual may also wear additional testing equipment, such as a heart monitor, pulse oximeter, etc. The captured air can then be analyzed to determined factors, such as fitness level, accumulation of lactose in the blood, metabolic efficiency, calories burned, etc. The data can serve as the basis for an individualized plan-for example, a medical treatment plan or suggested interventions such as behavioral changes.

By way of background, as a social process, value can be placed on those members of a community or population that have experience over expertise. Such value can be used to create an atmosphere or community where members can learn about others who have undergone the same interventions that an individual member is contemplating or in the process of doing. Additionally, the community can be used to help support the individual member, which can be a patient under the care of a physician or simply someone interested in the condition and/or disease.

Lifestyle change may be supported by online communities that provide support, ideas, and, in the case of these ideas, access to tools to provide objective data to make meaningful lifestyle changes in an individual user or members of a population. For example, in a community where a person is a peer, the actions they take and learn from are their volition and may result in increased agency (or autonomy) or self-efficacy. This not only increase the chances of continuous lifestyle improvement, but improved outcomes throughout the health system.

Thus, it is desirable to provide a community where members of the population can share interventions that worked for them and improved their metabolic testing results. Such a community can be implemented in a digital communication network (DCN) where members can interact with each other and the DCN. As one example, the community teaches and encourages members to conduct metabolic testing to determine if their respective resulting test points are improving. The community encourages individuals to share what they are doing and how it impacts their condition. Finally, the members of the community can use these experiences to find things they can try and share.

Further, online communities such as the DCN can be provided so that people can learn about healthy lifestyle practices and work to improve their health between clinical touchpoints, such as office visits. In many situations, patients can use their personal devices, such as a computer, tablet, cell phone, etc., to interact with other members in the DCN. The DCN can provide a platform for relaying communications, such as public posts, etc. and/or direct messages. Additionally, the DCN can store patient/member information, for example, biometric information, test results, personal data, etc. In some embodiments, the DCN system may provide services/apps, e.g., monitoring or even games.

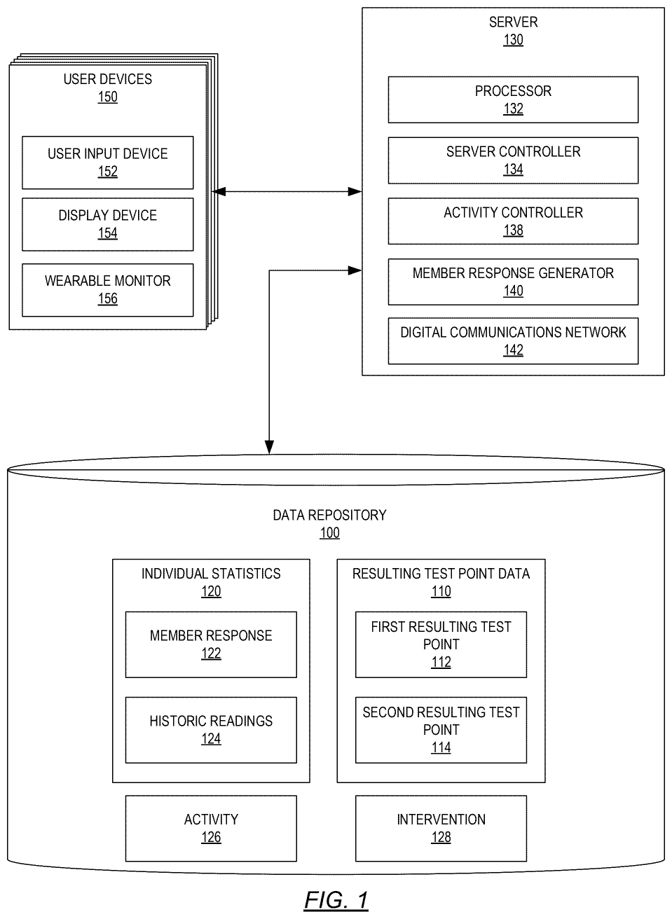

Attention is now turned to the Figures. The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores resulting test point data (110). The resulting test point data (110) includes readings from a biosample test, such as, for example, a metabolic test. The resulting test point data (110) may incorporate multiple readings taken over various times. For example, a first resulting test point (112) may be measured prior to an activity (126) (described below). Further, a second resulting test point (114) may be measured after the activity (126). In some embodiments, the resulting test point data (110) may include more or less resulting test points. For example, the resulting test point data (110) may include a third resulting test point, a fourth resulting test point, etc. The resulting test point data (110) may also include reading time data, which provides information regarding when various resulting test points are taken, such as the date and/or time a resulting test point is taken.

The resulting test point data (110) may be measured using, for example, saliva, blood, breath, and/or stool samples, as well as those from heart rate (HR), accelerometer samples, body temperature, skin conductivity, etc. Testing can also be done using psychometric instruments and EMA derived data. The resulting test point data (110) can also be measured from biosensors, a continuous glucose monitor, and/or wearable devices. The resulting test point data (110) may also be measured or based on user feedback provided to, for example, the DCN (142) via a user device (150). An individual body's physiological response to phycological, psychological, or mental stress can also be used to measure the resulting test point data (110). In at least one embodiment the resulting test point data (110) is an insulin response measured by, for example, testing an individual member's blood for glucose. Any measurements described above can be taken at home or at a clinic.

The data repository (100) also stores individual statistics (120). The individual statistics (120) may include personal information of an individual that can be used in determining their member response (122) (described below), such as height, weight, etc. This data can also include one or more previously calculated member responses (122) and historic readings (124) of the member responses (122).

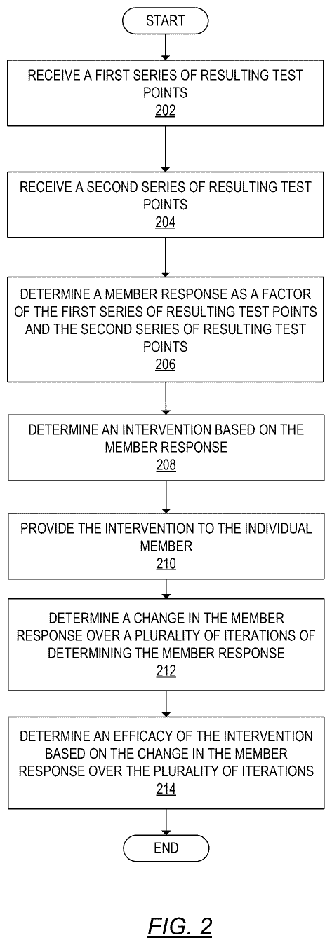

The data repository (100) also stores the member response (122). The member response (122) is the difference between resulting test points, such as the first resulting test point (112) and the second resulting test point (114) or the difference between a first series of resulting test points and a second series of resulting test points. The member response (122) can also be a function of the resulting test point data (110) and/or the individual statistics (120). The member response (122) can be measured by testing while using various activities, which will be described in detail in FIG. 2.

The data repository (100) also stores the activity (126). The activity (126) may be, for example, various digital stimulus or challenges, such as an adaptive game, a controlled encounter with another individual, therapeutic event, or an audial and/or visual stimulus (e.g., a movie, a piece of music or art). The activity (126) may also be a physical activity, such as, for example, running, climbing, or swimming. In such examples, the activity (126) may include instructions or prompts for completing the physical activity.

The activity (126) can also include instructing the individual member to have a drink or eat something when measuring insulin levels. In such examples, the drink or food may be a specific mixture to have a desired level of glucose moiety known to affect an individual member's glucose level. By linking the insulin level to the activity (126) that is known to have an impact on the insulin level measured, information about the sensitivity of the individual member's response to the activity (126) can be obtained.

In embodiments where the activity (126) includes ingesting an ingestible composition, the selection of the ingestible composition may be received from the individual member at DCN and sending, by the DCN, the selected ingestible composition to the individual member. The ingestible composition may be selected from a list of commercially available ingestible compositions. In some cases, the list of ingestible compositions are restricted to those containing a specific amount of glucose.

Similarly, in some embodiments, the activity (126) may be a game programmed to adapt to provide a constant degree of difficulty so as to induce a mental challenge and/or a psychological challenge to determine a stress level. By linking the stress level measurement to an activity (126) that is known to have an impact on the stress indicator level measured, e.g., measuring stress after an activity that is known to increase stress, information about the sensitivity of the individual's response to the activity (126) and the time to recover from the activity (126) can be obtained. Since the activity (126) is standardized (at least for the individual), it allows a new range of stress indicator level measurements to be used to encourage and measure the effects of behavior change. It also allows smaller changes to be seen which is often important in initiating behavior change to increase member response.

The data repository (100) also stores an intervention (128). The intervention (128) is generated for the individual member based on their member response (122) or a change in their member response (122). For example, a negative change in the individual member's member response will generate an intervention (128) that may provide suggestions to improve the individual member's member response (122). Such suggestions may include, for example, interacting with other members in a digital communication network (DCN) to learn how other members manage their insulin response, such as attending a seminar on improving or managing one's insulin levels or obtaining medical intervention. In some instances, the intervention (128) may be provided to the individual member's healthcare provider. In some embodiments, if the level of the individual member's member response (122) is positive, the intervention (128) may be to encourage the individual member to share their techniques for increasing and maintaining a positive level of member response (122).

In some embodiments, the intervention (128) may be provided to the population or sub-groups of the population. For example, a sub-group of members may have similar levels of member response (122) and the intervention (128) may be provided to the sub-group for suggestions on how to improve their levels of member response (122).

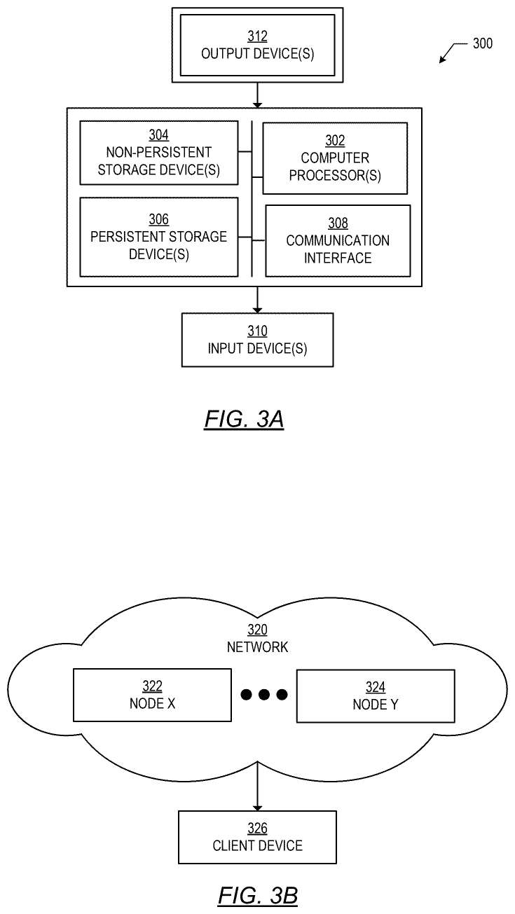

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as an activity controller (138) or a member response generator (140). An example of a computer system and network that may form the server (130) is described with respect to FIG. 3A and FIG. 3B.

The server (130) also includes a computer processor (132). The computer processor (132) is one or more hardware or virtual processors which may execute computer readable program code that defines one or more applications, such as the activity controller (138) or the member response generator (140). An example of the computer processor (132) is described with respect to the computer processor(s) (302) of FIG. 3A.

The server (130) also may include a server controller (134). The server controller (134) is software or application specific hardware which, when executed by the computer processor (326), controls and coordinates operation of the software or application specific hardware described herein. Thus, the server controller (134) may control and coordinate execution of the activity controller (138), and the member response generator (140).

The server (130) also includes the activity controller (138). The activity controller (138) is software or application specific hardware which, when executed by the computer processor (132) provides the activity as a digital stimulus or challenge to the user in order to generate a resulting test point (e.g., an insulin level, a stress response, etc.), such as through a game. In embodiments where the activity is a physical activity, the activity controller (138) may provide prompts to the individual to perform the activity.

The server (130) also includes a member response generator (140). The member response generator (140) is software or application specific hardware which, when executed by the computer processor (132), performs the method of FIG. 2. The member response generator (140) receives, as input, the resulting test point data (110) and determines the member response (122). In some embodiments, the member response generator (140) may also receive individual statistics (120) which are used in the generation of the member response (122).

The server (130) also includes the digital communications network (DCN) (142). The DCN (142) is a network through which members of a population can interact with each other, or with a system supported by the DCN (142). For example, an individual member can enroll for metabolic testing to determine their member response (122) through the DCN (142). In another example, the individual member can interact with other members to learn about techniques for improving their level of the member response (122).

In some embodiments, the DCN (142) can administer the activity to determine an individual member's member response (122). For example, if the activity (126) is a computer game, the DCN (142) can provide the computer game to an individual member. The DCN (142) can also be used to receive input from the individual member, such as measurements, feedback, or comments regarding the activity. For example, the individual member may use an application on their mobile device to send in the feedback or comments to the DCN (142), or may wear a wearable device or monitor (156) that can measure and send the measurements to the DCN (142).

The DCN (142) can also provide means for members of the population to communicate with each other. Members can share results from their member response testing or assessment and can also share techniques for improving their member response (122). The DCN (142) can also be used to deliver or transmit the intervention (128) to the individual member based on their member response (122).

In the DCN (142), patient messages may be ordered to prioritize messages from members whose level of member response (122) is higher or more positive over time. These communications between members of the population can be one-to-one, one-to-many, one-to-system, system-to-one, or system-to-many. The system may also feature an AI bot that derives its communications from analysis of communications in the digital communication network and/or biometrics provided to the DCN (142).

In another, non-limiting embodiment, patient messages in the DCN (142) may be ordered to prioritize messages from members who have had interventions that improve their member response (122). The interventions can be lifestyle behaviors and/or seeking professional care.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156) and be configured to send resulting test point data (110) to the server (130). In an alternative embodiment, a separate wearable device or monitor (156) may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method can be used to determine the member response of an individual member, and in some instances, generate an intervention to help the individual member improve their member response or encourage the individual member to share their activities and lifestyle when their member response is positive. Determining the member response for an individual member or a plurality of members of a population can provide a foundation for managing health care risk in the population.

In accordance with at least one embodiment the method includes an iterative process in which a member response for an individual member is determined. The iterative process can be repeated multiple times or executed one time.

At Step 202, a first series of resulting test points is received. In the first series, a first resulting test point (such as the first resulting test point (112)) is received prior to an activity (such as the activity (126)) and a second resulting test point (such as the second resulting test point (114)) is received after the activity. It will be appreciated that in some embodiments, more than one resulting test point can be received prior to or after execution of the activity. The activity may be administered by, for example, the DCN using an activity controller such as the activity controller (138).

The first series of resulting test points can be received by a digital communications network (DCN) such as the DCN (142) via a user device such as the user device (150). The activity, as previously described, is used to induce a change in the resulting test point of the individual member participating in the activity for the purpose of determining a member response such as the member response (122). The activity can be, for example, a normal life activity or food routinely consumed. The activity may be any activity that can be standardized, repeated, and induces a change in the member response in a known way.

The first resulting test point and the second resulting test point can be part of the resulting test point data such as the resulting test point data (110). The resulting test point(s) can be a measurement from a metabolic test. In some embodiments, the resulting test point(s) are a measurement of a stress hormone, such as, for example, cortisol and/or an enzyme, such as alpha-amylase. The stress indicator can be measured in saliva or capillary blood. In other instances, the resulting test point(s) can be a measurement of an insulin level of the individual member. The insulin level can be measured continuously or at time intervals by, for example, a glucose monitor.

In embodiments where the activity comprises ingesting an ingestible composition (to measure, for example, the insulin level), the method may also include receiving a selection of the ingestible composition from the individual at the DCN and sending, by the DCN, the selected ingestible composition to the individual. The ingestible composition may be selected from a list of commercially available ingestible compositions. In some cases, the list of ingestible compositions are restricted to those containing a specific amount of glucose.

The resulting test point data can be measured at home or at a clinic. The resulting test point data can be biometrics derived from saliva, or blood, or breath, or stool samples, as well as those derived from heart rate, accelerometer samples, body temperature, skin conductivity, as well as signals derived from wearables. It can also be obtained from psychometric instruments and emergency medical assistant derived data.

In some embodiments, the Step 202 may include receiving additional resulting test points at different time points after the activity. For example, a third resulting test point may be obtained after the second resulting test point, a fourth resulting test point may be obtained after the third resulting test point, etc.

At Step 204, a second series of resulting test points are received. The second series of resulting test points include a first resulting test point measured prior to the activity and a second resulting test point measured after the activity. The activity in the Step 204 is the same activity in the Step 202.

The second series of resulting test points can also be received by the DCN via the user device. In some embodiments, the Step 204 may also include receiving additional resulting test points at different time points after the activity. For example, a third resulting test point may be obtained after the second resulting test point, a fourth resulting test point may be obtained after the third resulting test point, etc.

At Step 206, a member response is determined as a function of the first series of resulting test points and the second series of resulting test points. The member response can be determined by and received as output from a member response generator such as the member response generator (140). For example, the member response can be determined by the member response generator (140) as a function of the time it takes for the second resulting test point and the third resulting test point (e.g., the resulting test points after the activity) to return to the range of the first resulting test point (e.g., the resulting test point prior to the activity) for a given series. In other examples, the member response can be calculated as a function of the slope of the second resulting test point and the third resulting test point (or any number of post-activity resulting test points) for a given series. Alternatively, the resulting test point can be calculated as an index of a function of the difference between the first resulting test point and both the second resulting test point and the third resulting test point, the time it takes for the second resulting test point and the third resulting test point to return to the range of the first resulting test point, and a function of the slope of the second resulting test point and the third resulting test point for a given series.

At Step 208, an intervention such as the intervention (128) based on the member response is determined. As previously described, the intervention may be determined or generated based on the member response, as determined in the Step 206. For example, in instances where the member response is determined to be negative, the intervention may provide suggestions to improve the individual member's member response. In another example, when the member response is determined to be positive, the recommendation may provide suggestions for the individual member to share their techniques for maintaining a positive member response with other members through the DCN.

At Step 210, the intervention is provided to the individual member. The intervention may be provided to the user from the DCN via the user device. In other embodiments, the intervention may also be provided to the user from any device or system.

At Step 212, a change in the member response is determined over a plurality of iterations of determining the member response (e.g., a plurality of iterations of the steps 202-210). The change in the member response may simply be a difference between the member response at different iterations. In other embodiments, the member response may be averaged over the different iterations.

At Step 214, an efficacy of the intervention is determined based on the change in the member response over the plurality of iterations. In some embodiments, the efficacy may be determined based on a threshold. For example, if the change in the member response is below the threshold, then this may indicate that the intervention provided to the user was ineffective. This may result in adjustments or changes to the intervention. In another example, if the change in the member response is above the threshold, then this may indicate that the intervention provided to the user was effective. In such example, the intervention may not be changed for the user.

The method of FIG. 2 described above may have more or less steps than shown. Further, steps may be repeated as needed. For example, the Steps 202, 204, 206, 212, and 214 may be repeated to evaluate if the intervention provided in the Step 208 and 210 was effective.

A specific example of the above method of FIG. 2 will now be described. In such example, the resulting test point data measured is an insulin level of an individual member and such insulin level can be measured by testing the individual member's blood for glucose. The individual member's glucose level may be measured prior to and after an activity. The activity may be, for example, ingesting a food having a known glucose moiety.

In such example, a first series of insulin levels may be measured prior to and after the individual member has ingested a first portion of the food. A second series of insulin levels may then be measured prior to and after they have ingested a second portion of the food. In such examples, the food ingested for the first series of insulin levels and the second series of insulin level is the same and a time period between the insulin level measured prior to and after the individual member has ingested the food is also the same. The first series of insulin levels and the second set of insulin levels may then be compared to measure a change in the insulin response of the individual member.

In the same example, comparing results of the first series and the second series includes comparing a difference in an area under a curve of the insulin values for the first series and the second series. The area under the curve may be based on peak insulin value, or insulin value at a specific time point. Further, the example may also include collecting at least one additional series of insulin levels at a subsequent time and comparing results of the first series and the second series, this also includes comparing the at least one additional series.

In another specific example of the above method of FIG. 2, the resulting test point data measured is a stress level. In the specific example, the activity may be a video game known to induce stress in an individual. A first stress level can be obtained from the individual prior to starting the video game. Then, a second stress level can be obtained from the individual after the individual has completed the video game. A stress resiliency of the individual can then be determined based on the first stress level and the second stress level

In such example, a level of the stress resiliency may be below a threshold, which indicates that the individual has a low level of stress resiliency. Thus, a recommendation may be generated and provided to the user that suggests the individual to interact with other members of a DCN having the same or similar stress resiliency results. The recommendation may also suggest that the individual share their reflections on their stress resiliency with other members of the DCN. The recommendation to interact with other members of the DCN may be provided to the individual by the DCN via a user device that the individual owns, such as a smartphone, laptop, wearable device, etc.

The individual may then have their stress resiliency redetermined by participating in the activity again and having their first stress level and their second stress level remeasured. A change in the individual's stress resiliency indicates that the recommendation was either effective or ineffective. For example, if the individual's stress resiliency increases, then the recommendation may be determined to be effective. If the individual's stress resiliency decreases, then the recommendation may be determined to be ineffective.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN), such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302). Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium, such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to determine a member response in an individual member, the method comprising:
determining a member response by:
receiving a first series of resulting test points including a first resulting test point measured prior to an activity and a second resulting test point measured after the activity;
receiving a second series of resulting test points including a first resulting test point measured prior to an activity and a second resulting test point measured after the activity; and
determining the member response as a function of the first series of resulting test points and the second series of resulting test points;
determining an intervention based on the member response;
providing the intervention to the individual member;
determining a change in the individual member over a plurality of iterations of determining the member response; and
determining an efficacy of the intervention based on the change in the member response over the plurality of iterations.

2. The method of claim 1, wherein the first resulting test point and the second resulting test point comprise a first insulin level and a second insulin level, respectively.

3. The method of claim 1, wherein the first series of resulting test points and the second series of resulting test points are measured using a continuous glucose monitor.

4. The method of claim 1, wherein the activity comprises instructing the individual member to ingest at least one of food or a fluid.

5. The method of claim 4, wherein the food or the fluid comprises a mixture of a glucose moiety.

6. The method of claim 1, wherein the member response is calculated as a function of the difference between the first resulting test point and the second resulting test point.

7. The method of claim 1, further comprising receiving a third resulting test point, and wherein the member response is determined as a function of the time taken for the third stress resulting test point to return to the range of the first resulting test point.

8. The method of claim 7, wherein the member response is determined as a function of a slope of the second resulting test point and the third stress indicator level.

9. The method of claim 7, wherein the member response is calculated as an index of a function of the difference between the first resulting test point and both the second resulting test point and the third resulting test point, the time it takes for a fourth resulting test point taken after the third resulting test point to return to the range of the first resulting test point, and a function of a slope of both the second resulting test point and the third resulting test point.

10. The method of claim 1, wherein measuring the first resulting test point and the second resulting test point comprises taking a reading using at least one of: a psychometric instrument, a heart rate monitor, and a pulse oximeter.

11. The method of claim 1, wherein measuring the first resulting test point and the second resulting test point comprises sharing the first resulting test point and the second resulting test point using the DCN.

12. A method to determine member response in an individual member, the method comprising:
determining the member response by:
receiving a first resulting test point measured prior to an activity;
receiving at least a second resulting test point and a third stress indicator level measured at two or more time points after the activity, wherein the activity is operable to induce a stress response; and
determining the member response as a function of the first measured stress indicator level, the second measured stress indicator level, and the third measured stress indicator level,
wherein the first resulting test point, the second resulting test point, and the third stress indicator level form a plurality of stress indicator levels;
determining an intervention for an intervention based on the member response; and
providing the intervention to a user.

13. The method of claim 12, wherein the method is performed at multiple instances, and
wherein the method further comprising determining a change in the member response over multiple iterations of the method.

14. The method of claim 13, further comprising determining an efficacy of an intervention based on the change in the member response over the multiple instances.

15. The method of claim 12, wherein the member response is calculated as a function of the difference between the first resulting test point and both the second resulting test point and the third resulting test point.

16. The method of claim 12, wherein the member response is determined as a function of the time taken for a fourth resulting test point taken after the third resulting test point to return to the range of the first resulting test point.

17. The method of claim 12, wherein the member response is determined as a function of a slope of the second resulting test point and the third resulting test point.

18. A system comprising:
a computer processor;
a data repository in communication with the computer processor and storing:
individual statistics having a member response and historic readings,
resulting test point data having at least a first resulting test point and a second resulting test point,
an activity, and
an intervention;
an activity controller which, when executed by the computer processor, administers the activity;
a member response generator which, when executed by the computer processor, determines the member response;
a digital communications network which, when executed by the computer processor, provides a network for members of a population to interact with each other and for an individual member to access a metabolic test for determining the individual member's member response;
a server controller which, when executed by the computer processor:
determines the member response by:
receiving a first series of resulting test points including a first resulting test point obtained prior to the activity and a second resulting test point after the activity,
receiving a second series of resulting test points including a first resulting test point obtained prior to the activity and a second resulting test point after the activity,
determining a member response as a function of the first series of resulting test points and the second series of resulting test points, determines an intervention for an individual based on the member response;
provides the intervention to the user;
determines a change in the member response over a plurality of iterations of determining the member response; and
determines an efficacy of the intervention based on the change in the member response over the plurality of iterations.

19. The system of claim 18, wherein the first resulting test point and the second resulting test point are a first insulin level and a second insulin level.

20. The system of claim 18, wherein the activity comprises instructing the individual member to ingest at least one of food or a fluid.
